(19) United States
(12) Patent Application Publication
(10) Pub. No.: US 2008/0220513 A1
(43) Pub. Date: Sep. 11, 2008

(54) Device and method for collecting of organic waste

(76) Inventor: Paul A. Bucci, (US)

Correspondence Address: ASMISH G. AIRAN, 814 LOVE LANE, WARWICK, RI, 02818-4032, US

(21) Appl. No.: 12/011,788

(22) Filed: Jan. 31, 2008

Related U.S. Application Data
(63) Continuation-in-part of application No. 11/604,532, filed on Nov. 27, 2006.

(51) Int. Cl. C12M 1/00

(52) U.S. Cl. 435/290.1

(57) ABSTRACT

A device and method for collecting organic waste is disclosed. The invention comprises a biodegradable bag that hangs inside a rigid container covered by a perforated lid. A filter lines the inside of the lid which rests on the rim of the rigid container. The biodegradable bag is secured onto an annular flange by means of a retaining band. The flange, together with the biodegradable bag, sits on the shoulder on the inside of the rigid container so that the biodegradable bag hangs from the flange leaving a cavity inside the rigid container between the biodegradable bag and the walls of the rigid container. The flange has a handle to pull the biodegradable bag out. The biodegradable bag aids in composting of the organic waste as it is permeable to both air and water and provides the largest possible surface area of any waste container to aerate the organic waste.

### CROSS-REFERENCE TO RELATED APPLICATIONS

This is the first Continuation-in-Part application of the parent application no. U.S. Ser. No. 11/604,532 filed on Nov. 27, 2006 and which was titled “A device and method for collecting of organic waste.”

This application claims the benefit of the earlier filed parent application number U.S. Ser. No. 11/604,532 under 37 CFR 1.53(b) and hereby incorporates that earlier filed parent application by reference. This CIP application claims an improvement over the earlier filed parent application no. U.S. Ser. No. 11/604,532.

### STATEMENT REGARDING FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISC APPENDIX

Not applicable

### BACKGROUND

1. Field of Invention

Current invention is directed towards a device and a method for collecting organic waste that can eventually be converted into compost. It is well known in the composting industry that organic waste that is readily compostable may include kitchen waste such as plant and food leftovers and vegetable peelings, tea leaves, coffee grounds, household waste such as weed, yard leaves, grass or tree/shrub prunings, waste paper; however, this is not an exhaustive list of all possible compostable material.

Presently, most municipalities separately collect solid waste and recyclable waste like plastic, glass and paper. The current invention is directed towards reducing solid waste by offering a third option of collecting all household waste that contains fluids and is organic. Anaerobic decomposition of the fluid containing organic waste by bacteria and fungi causes putrefaction resulting in foul odor and may even produce toxic fumes like methane and is an active breeding ground for disease causing organisms. Aerobic decomposition tends to produce fewer toxic fumes. For this reason, any device or a method that can promote aerobic decomposition is preferable.

Proper techniques can promote aerobic decomposition utilizing the microorganisms that are already present in the said organic waste converting the fluid containing organic waste into environmentally friendly compost. The current invention attends to the genuine desire among environmentally conscious households to reduce the amount of waste produced that consumes large tracts of land as landfills. Additionally, the present invention improves the method, employing which one can recycle the organic waste into compost in a simple, clean and odorless manner. This device is economical to manufacture, portable and if desired, can easily be kept indoors without any concern for offensive odors.

Finally, waste management corporations can adopt the organic waste collecting method introduced in this invention to recycle municipal organic solid waste. For example, municipalities can use this invention in public areas such as public parks. Just as presently public parks contain especially marked bins for collecting glass bottles and aluminum cans, similarly especially marked bins can be employed to collect organic waste. And from the time that the waste is collected to the time that it is disposed of in a compost pile, the present invention will promote aerobic decomposition of the said organic waste so that its eventual disposal in the compost pile will result in faster compost formation in the preferred less toxic fashion.

2. Description of Prior Art

Composting is aerobic decaying of organic waste wherein a myriad of aerobic and other microorganisms chemically decompose cellulose present in the organic waste to produce compost over time. Consequently, production of compost is enhanced by and requires proper aeration and optimal moisture content of the said organic waste heap. Poor air circulation or presence of excessive water in the organic waste promotes anaerobic decomposition and putrefaction resulting in offensive and potentially harmful odors.

Unlike the present invention, most inventions in the field of composting seek to convert organic waste into compost by holding the said waste in a container for a specified amount of time and provide a means to aerate the said waste. Some of these devices employ elaborate and complex systems containing multiple and sometimes movable parts, which make them too complex for widespread adoption by the consumers. Inherent drawback in the design of this genre of composting bins is the fact that the means for aeration exposes only a limited surface area of the organic waste contained in a bin to aeration.

Collinson, in U.S. Pat. No. 5,178,469 (hereinafter referred to as “the '469 patent”), discloses a collapsible biodegradable container bag for liquid-containing solid materials. Unlike the present invention, the '469 patent is “liquid impervious” and necessarily teaches a rectangular cross section for the device to work as described in the specification. The '469 patent further contains a “rectangular liquid-absorbent lining adhesively attached” to the inside of the bottom wall. Finally, the '469 patent uses a pure cellulose film as a means to restrict the passage of water. The current patent does not have any mechanism to absorb the liquid and does not have any limitation as to the shape of the biodegradable bag or the rigid container. Instead, as described below, the present invention depletes the contents of the liquids present in the organic waste and collects the liquids into a rigid container underneath a biodegradable bag.

In U.S. Pat. No. 5,403,740 (hereinafter referred to as “the '740 patent”), Menefee & Menefee also disclose a collapsible, biodegradable compost bin. The primary difference between the bin in '740 patent and the present invention is that the '740 patent suggests a bin “substantially cylindrical in shape to minimize the surface area of the bin for a given volume.” However, as is described below, the present invention seeks to maximize the surface area of the organic waste contained in a biodegradable bag that is exposed to air inside the rigid container. Additionally, unlike the present invention, in the '740 patent the whole bin is biodegradable and it gives specific guidelines as to the construction material of the bin.

Some devices, for example, U.S. Pat. No. 5,843,768 awarded to Lin & Knock and Morrison patent application US2002/0081717 A1 (hereinafter referred to as “the Morrison patent application”), predominantly include a container with front, rear and side walls enclosing at least one large chamber and one small chamber. The large chamber at the top of the container is used as a repository for organic waste to be decomposed and the second small chamber located below the large chamber is separated via a perforated divider that presumably allows the liquids to escape from the organic waste in the upper large chamber into the lower small chamber.

This genre of devices also includes one or more aerator composed essentially of a tube with multiple apertures and that passes through the organic waste. The air inside the cavity of this tube is generally in communication with the outside environment. Such a device will spread the foul odor around the device and also would attract undesirable pests. The Morrison device also discloses a bag lining. However, the disclosed function of that bag is to insulate the decomposing material, and to assist in the emptying of the contents of the container. Thus in the Morrison patent application, the function of the bag is not to aerate the contents. This bag has an opening at the bottom to permit an aerator tube to pass through the waste that it collects. Consequently, in order to remove the contents of the bag, the Morrison patent application teaches to invert the bag and remove the material from the bottom of the bag. In order to prevent falling of material when the bag is inverted, the Morrison patent application teaches to employ a drawstring on both ends of the bag. This act of removing the bag to pull the aerator tube and apply a drawstring is highly undesirable and potentially unpleasant experience as it presents a very likely possibility of spilling the contents of the bag. The present invention overcomes these drawbacks and offers a more pleasant way of emptying the container when it is full.

### SUMMARY OF THE INVENTION

The present invention is directed towards a device and a method for collecting compostable fluid containing organic waste. This invention comprises of a rigid container, a biodegradable bag or net that aerates and gradually depletes the organic waste of liquids present in the said organic waste, a perforated lid that covers the rigid container, a flange onto which the biodegradable bag is fastened with the help of a retaining band. The face of the lid that is in direct contact with the inside of the rigid container is lined with a filter. When the biodegradable bag, which is a repository for organic waste, gets full, a handle on the flange is used to pull the biodegradable bag together with the flange, out of the rigid container. The current invention promotes optimal aeration of the organic waste resulting in aerobic oxidation of the said waste. This design offers maximum possible aeration of the organic waste as compared to any other design and without the need for undesirable and sometimes unpleasant periodic manual tilling of the said organic waste and without the need for complex construction. The present invention holds the organic waste only until it is completely filled after which time the biodegradable bag containing the organic waste is deposited into a compost pile. The present invention is thus a simple device, economical and easy to manufacture and easy to use—all desirable qualities of a novel device that may lead to its widespread use in today's environmentally conscious society.

A new alternate embodiment with insect trap has a lid geometry that creates an insect trap and an empty space for insect collection. The empty space is created between the underside of the lid and the top or upper side of the filter. This embodiment of the invention allows small disease spreading bugs, flies, fruit flies, insects, etc. to enter the trap however they are not able to escape. The dead insects collect on the filter and can later be discarded.

3. Advantages of Current Invention

In the current invention, the primary receptacle of organic waste is a bag manufactured from a biodegradable, mesh like material and hangs inside a rigid container on a flange secured by a retaining band. Just like any other compliant or flexible bag, this biodegradable bag takes different shapes depending on its contents. This construction and use of the biodegradable bag promotes optimal aeration of the organic waste resulting in aerobic oxidation of the said waste. This design of the organic waste collecting device offers maximum possible aeration of the organic waste as compared to any other organic waste collecting bin without the need for undesirable and sometimes unpleasant periodic manual tilling of the said organic waste.

Moreover, the biodegradable mesh bag ensures that the liquids from the organic material called leachate flow into the inside cavity of the rigid container. And since the leachate sits in close proximity with the biodegradable bag above, the organic waste does not loose too much water and become dehydrated. If, for example, the contents of the biodegradable bag become too dry, the waste will absorb the water vapor from the leachate, prompting more evaporation of the water from the leachate. If, however, the water content of the organic waste inside the biodegradable bag is too high, more water will simply leach into the bottom of the rigid container via the action of gravity.

The new alternate embodiment with insect trap has the added advantage of being able to catch and trap disease spreading bugs, flies, fruit flies, insects, etc. The dead insects collect on the filter.

Once the container is full, this invention allows the user to simply lift handle on the flange to pull the biodegradable bag that is secured on the flange via a retaining band, out of the rigid container and deposit the biodegradable bag containing the organic waste into a compost pile. Since the bag is manufactured from inexpensive biodegradable mesh, the bag itself will decay into compost. Thus, the device is user friendly and offers the ease of operation.

### BRIEF DESCRIPTION OF DRAWINGS

FIG. 1 is an exploded view of the preferred embodiment of the invention.

FIG. 2 is the view from top (with the lid) of the preferred embodiment of the invention.

FIG. 3 is a sectional view.

FIG. 4 is sectional view of the lid—an alternate embodiment with insect trap.

FIG. 5 is sectional view of the lid with insect trap—an alternate embodiment.

### REFERENCE NUMERALS

The following is a list of numerals and their respective references as used in the attached drawings:
1. Lid
2. Filter
3. Rigid container
4. Flange
5. Biodegradable drainage bag
6. Retaining band
7. Handle
8. Inside cavity
9. Shoulder
10. Rim (in which the Lid rests)
11. Venting holes
12. Knob
13. Raised ridge of flange
14. Alternate Embodiment: Extra space between the Lid (1) and the Filter (2)
15. Alternate Embodiment: Raised lid (1) with sidewalls
16. Alternate Embodiment: L-shaped sidewall

### PREFERRED EMBODIMENT

### Detailed Description

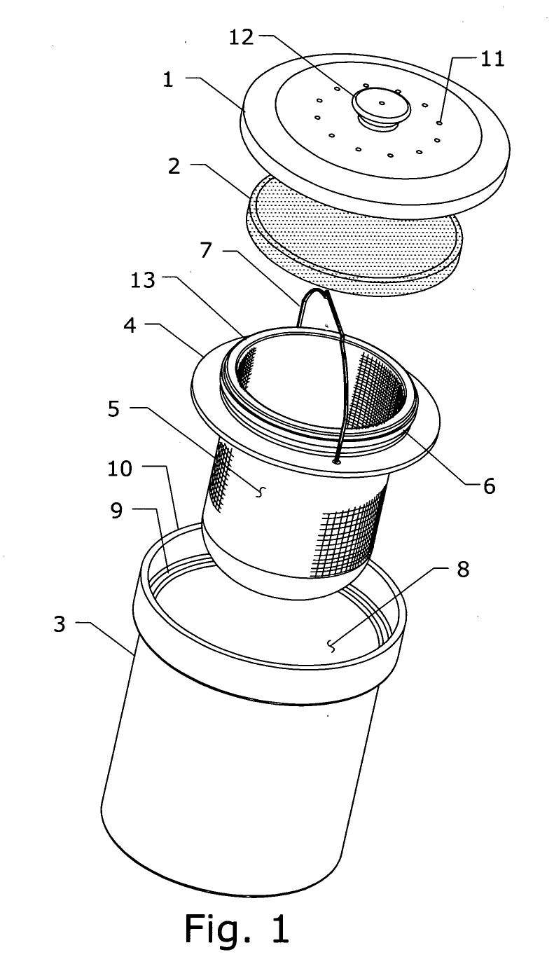

FIGS. 1-3 illustrate preferred embodiment of the invention. FIG. 1 is an exploded view of the preferred embodiment and illustrates a rigid container (3) enclosing an inside cavity (8) with an opening at top; said container (3) having rim (10) and a shoulder (9), both located close but parallel to each other towards the top of the said rigid container (3). The rim (10) is designed to fit the lid (1) so that said lid does not fall into the inside cavity (8) of the said rigid container (3); at the same time the said lid (1) covers the contents of the rigid container (3). The lid (1) can be lifted by holding the knob (12). The dimensions of these individual parts are proportional to the overall size of the device.

In the preferred embodiment of the invention, the shoulder (9) is designed to hold an annular flange (4). Flange (4) suspends a biodegradable bag (5) which is secured by a retaining band (6). The flange (4) has a handle (7) having a storage “down” position and a functional “up” position. The handle (7) is used to lift the biodegradable bag (5) out of the rigid container (3) when the said biodegradable bag (5) has been filled with organic waste. The annular flange (4) has a broad surface that is constructed from a material designed to support the anticipated weight of the completely filled biodegradable bag (5). The inside edge of the flange (4) has a raised ridge (13). In order to hang the biodegradable bag (5), the top of the biodegradable bag (5) goes over the ridge (13) of the annular flange (4) and a retaining band is tightened around both the biodegradable bag and the raised ridge (13) of the annular flange (4) such that the retaining band (6) is tight enough so that the biodegradable bag (5) does not slip out from between the said retaining band (6) and the raised ridge (13) of the said annular flange.

The biodegradable bag (5), which is manufactured from a mesh like biodegradable and environmentally friendly material, aerates the organic waste while at the same time allowing any excess liquids present in the organic waste to escape through its pores into the inside cavity (8) of the said rigid container (3). The size of the biodegradable bag (5) is such that when it is suspended from the flange (4) with the help of the retaining band (6), it maintains a gap large enough so that the top meniscus of the leachate at the bottom of the rigid container (3) does not touch the bottom of the biodegradable bag (5); This gap also allows for some natural but limited expansion of the biodegradable bag (5) due to the weight of the organic waste.

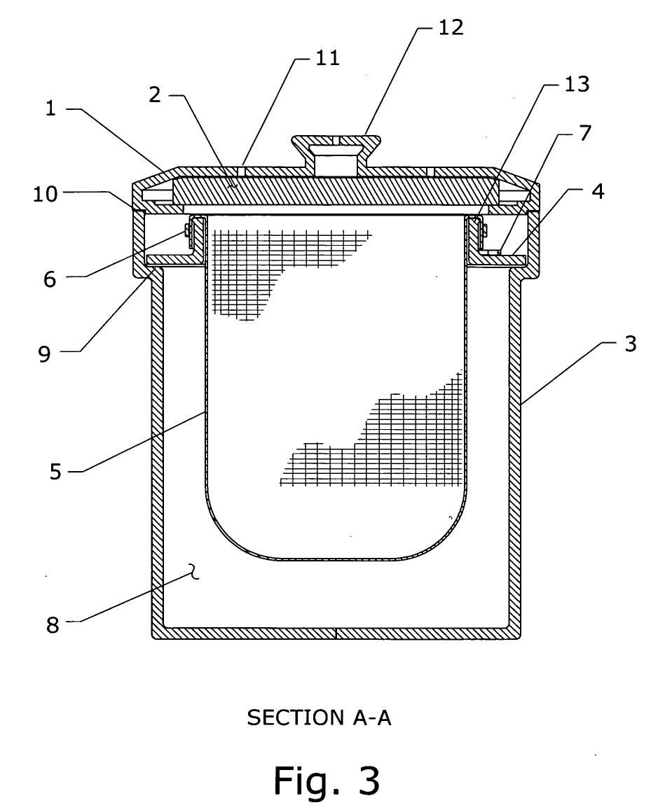

When in use, the biodegradable bag (5) acts as a receptacle for the organic waste. As with other parts of this device, the shape and size of the biodegradable bag (5) will correspond to the shape and size of the rigid container (3). FIG. 3 is a sectional view of the preferred embodiment. Although in FIG. 3, which is a vertical cross section of the device, the rigid container appears rectangular, it is recognized that in practice the container can be variously ornamentally shaped. It is, however, required that the said rigid container (3) has rigid walls so that it does not collapse from the weight of the organic waste in the biodegradable bag (5).

Conversely, since the biodegradable bag (5) is manufactured of non-rigid material that will take a new shape each time it is filled with organic waste, the opening at the top of the said container must be large enough so that organic waste can be conveniently placed into the biodegradable bag (5) and as needed, said biodegradable bag (5) can be conveniently removed from the said rigid container (3) when the said biodegradable bag (5) is full of organic waste.

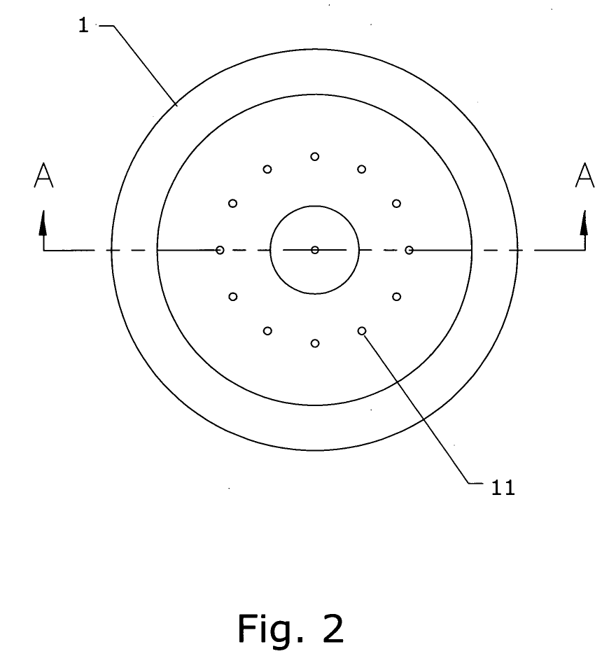

As shown in FIG. 2, the lid (1) is has venting holes (11) so that the inside cavity (8) and the contents of the biodegradable bag (5) are in constant communication with the outside atmosphere to allow the aeration of the organic waste. The underside of the lid (1) contains a filter (2) to moderate any possible odor emanating from the device. This filter also deters convergence of insects and other undesirable organisms near or around the device.

In an alternative embodiment of this device, the bottom of the rigid container (3) may contain an optional drain to allow excess liquids to escape if the organic waste contains excess liquids that accumulate at the bottom of the rigid container (3) and risks contact with the bottom of the biodegradable bag (5). To accurately see the level of the leachate and the bottom of the suspended biodegradable bag (5), the bottom of the rigid container (3) may be manufactured from a transparent material. The level of the leachate can then simply be estimated by looking through that transparent material. If the level of the leachate is too close to the bottom of the biodegradable bag, then the said biodegradable bag may be lifted out to discard some of the leachate.

### ALTERNATE EMBODIMENT WITH INSECT TRAP

### Detailed Description

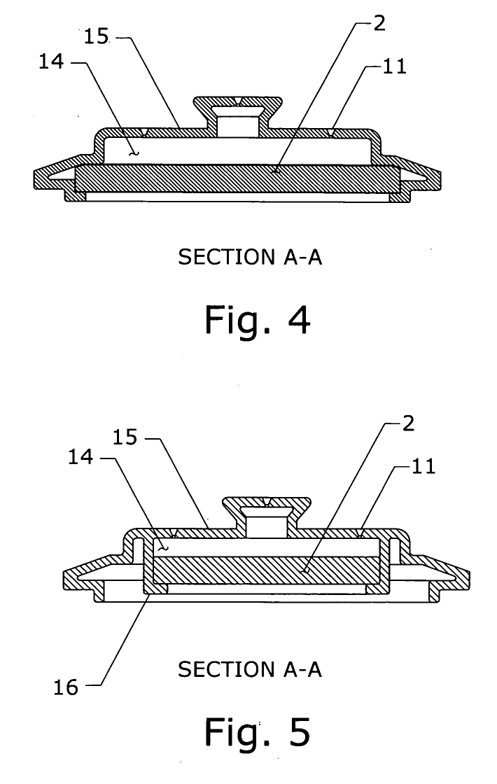

FIG. 4 illustrates an alternate embodiment with the insect trap. As mentioned above, one of the biggest deterrents to widespread adoption by the environmentally conscious public of this composting technology is the undesirable side effect of the odor emanating from composting waste that attracts undesirable insects. Although the preferred embodiment that is mentioned above, reduces the amount and intensity of the odor emanating from the preferred embodiment by using a filter, this alternate embodiment improves upon that preferred embodiment as follows:

This alternate embodiment introduces an empty space between the lid (1) and the filter (2) to trap undesirable insects and bugs. This entrapment of the undesirable insects and bugs together with reduction in odor addresses the biggest concerns of the environmentally conscious and the general public and will perhaps promote widespread adoption this composting technology by them.

In this alternate embodiment, an empty space (14) between the lid (1) and the filter (2) is created by raising a portion of the lid upwards, such that the underside of the lid (1) and a portion of the top or upper side of the filter (2) are in contact with each other only around the perimeter of the filter. It is imperative to maintain this contact so that the trapped insects do not fly into the rigid container (3). As shown in FIG. 4, the sidewall of the lid (1) is shaped like “L” so that the filter “snaps into” and rests on the “small” arm” of “L” shaped sidewall. Thus, the empty space (14) is enclosed by the underside of the lid (1), the top of the filter (2) and the side of the lid (1) that is in contact with the perimeter of the filter (2).

The empty space (14) is in contact with the outer environment through venting holes (11) through which the insects can then enter the empty space (14). Any insects that enter the empty space (14) either stay on the filter (2) or in the empty space (14). The size of the venting holes (11) on the lid (1) can be varied or modified depending on the size of the insect that need to be trapped. If the lid (1) is constructed from a thick material, the holes in the lid (1) can be shaped like a funnel wherein the holes have a larger diameter on the surface of the lid (1) that is exposed to the environment and a smaller diameter on the inside surface of the lid (1) that is exposed to the interior of the rigid container (3). These funnel shaped holes are best suited to trap the insects in the empty space (14).

If the lid (1) is constructed from a thin material, holes with one diameter will also work. The insects are able to detect and are attracted by the smell emanating from a composting device. These holes allow the insects to enter the container however the insects are then unable to escape.

Another method of creating the empty space (14) is to attach a filter encasement to the underside of the lid (1). As shown in FIG. 5, this filter encasement is created by attaching “L” shaped sidewall (16) to the underside of the lid (1). The filter (2) rests on the “small arm” of the “L” shaped sidewall (16). Thus, the empty space (14) is enclosed by the underside of the lid (1), the top of the filter (2) and the new “L” shaped sidewalls. In this case, the venting holes (11) are present only on that portion of the lid (1) underneath which the filter encasement is attached so that the trapped insects do not escape into the rigid container (3).

### PREFERRED EMBODIMENT

### Operation

When the device is in use, it is assembled as shown in FIGS. 1-3 and as described in PREFERRED EMBODIMENT: DETAILED DESCRIPTION above. When the assembled device is empty, air reaches the inside cavity of the rigid container through the pores in the mesh like material of the biodegradable bag. The organic waste is deposited into the biodegradable bag by lifting the lid that is lined with a filter. As the lid is lifted to deposit more organic waste, the organic waste that is already present in the device is exposed to air in the outside environment. Additionally, air that is present in the inside cavity of the rigid container is exchanged with the outside air through the mesh like material of the biodegradable bag that is not yet covered by organic waste. In this manner, along with the gradual exchange of air through the filter in the lid, the organic waste contained in this device is exposed to air from the outside environment. As compared to other devices on the market that expose the organic waste to air through aerated tubes, the current invention exposes the entire surface of the organic waste that is directly in contact with the biodegradable bag to the outside air.

The second function of the mesh like material of the biodegradable bag is to deplete the organic waste of fluids into the bottom of the rigid container. The liquid that accumulates at the bottom of the rigid container is called leachate. By design, the leachate sits in close proximity with the biodegradable bag above. If the contents of the biodegradable bag become too dry, the waste will absorb water vapor from the leachate. If, however, the water content of the organic waste inside the biodegradable bag is too high, more water will simply leach into the bottom of the rigid container via the action of gravity. In this manner, this device maintains an optimum level of moisture content in the organic waste while at the same time exposing maximum possible surface of the organic waste to air. Together these two functions of the device promote aerobic decomposition of the said organic waste so that its eventual disposal in the compost pile will result in faster compost formation.

This device is highly portable and can be placed outside or inside a house to collect the organic waste. If placed inside, once the biodegradable bag is full, the device can be taken outside and the biodegradable bag can be lifted out of the rigid container together with the flange with the help of the handle on the flange. The biodegradable bag can then be released from the flange after releasing the retaining band and the top of the bag can be tied with a commonly available tie, if needed. Since all the fluids from the organic waste have already leached into the rigid container, the biodegradable bag should not drip when lifted out from the rigid container. The biodegradable bag containing the organic waste can then be deposited into a compost pile and the leachate in the rigid container can simply be discarded by pouring it out of the rigid container.

Finally, the third function of the biodegradable bag is to biodegrade as and when it is deposited into a compost pile. Since the bag is made from inexpensive biodegradable material, the cost of operation of this device is low and the total ease of operation is high. In the drawings, the rigid container is shown as cylindrical in shape. However, this device can be manufactured in various sizes and can also have various ornamental shapes including but not limited to rectangular or square shapes. Similarly, the shape of the biodegradable bag will correspond to the shape of the rigid container. It can also be manufactured from several materials like plastic or ceramic. Thus, can be designed to suit the taste and needs of the user.

I claim:

1. An organic waste collecting device apparatus comprising:
an air and water proof rigid container having a rim with a shoulder running parallel to the said rim and located towards the top opening of the said rigid container;
a perforated lid with a knob that rests on the said rim of the said rigid container to cover the contents inside the said rigid container;
a filter that rests on that side of the said lid that is exposed to the inside of the said rigid container and that is opposite to the side that contains the said knob, which is used to lift the said lid;
a rigid annular flange with a handle and a raised ridge; said flange resting on the shoulder of the said rigid container and said handle having an operational up and a storage down position;
a compliant or flexible bag constructed from a mesh like biodegradable material for collecting fluid containing organic waste and for hanging from the said flange by going over the raised ridge of the said flange and secured on the said raised ridge of the said flange by means of a retaining band, said retaining band fitting the said raised ridge of the flange such that when the said retaining band is tight and operational, the said biodegradable bag does not slip out from between the raised ridge of the said flange and the said retaining band.

2. A method of collecting organic waste by means of an organic waste collecting device apparatus as claimed in claim 1 and including the steps of
depositing fluid containing organic waste into a compliant or flexible bag constructed from a mesh like biodegradable material for collecting the said organic waste and hanging inside a rigid container from a support means that can support the weight of the biodegradable bag when full and the material of the said biodegradable bag further having the following properties:
such bag constructed from a material that additionally allows aeration of the whole surface of the said organic waste that is in contact with the walls of the said biodegradable bag through the pores in the material of the said biodegradable bag and further allowing the leachate to leach out through the pores in the said biodegradable bag into the inside cavity of the said rigid container;
collecting the leachate that has escaped through the pores of the biodegradable bag, into the inside cavity of the said rigid container wherein the size of the biodegradable bag is such that when it is suspended from the said support means it leaves a gap large enough for some natural but limited expansion of the biodegradable bag due to the weight of the organic waste and large enough so that the biodegradable bag does not touch the top of the leachate surface below;
a means to cover the top of the rigid container with a lid lined on the inside with a screen or a filter to deter convergence of insects and other undesirable organisms near or around the device.

3. An organic waste collecting device apparatus as claimed in claim 1 in which a lower portion of the rigid container is manufactured from a rigid transparent material to allow the user to estimate the level of the leachate inside the said rigid container.

4. An organic waste collecting device apparatus as claimed in claim 1 further comprising:
an empty space between the lid and the filter, said empty space created by raising a portion of the lid upwards, such that the underside of the lid and a portion of the top or upper side of the filter are in contact with each other only around the perimeter of the filter and said empty space being enclosed by the underside of the lid, the top of the filter and the sidewalls of the lid that follow the perimeter of the filter;
the sidewalls of the lid shaped like “L” so that the filter rests on the “small arm” of the L-shaped sidewall;
funnel shaped holes in the lid manufactured from a thick material wherein the holes in the lid have a larger diameter on the surface of the lid exposed to the environment and a smaller diameter on the surface exposed to the inside of the rigid container.

5. (canceled)
